(19) United States
(12) Patent Application Publication
(10) Pub. No.: US 2019/0303758 A1
(43) Pub. Date: Oct. 3, 2019

(54) RESOURCE ALLOCATION USING A LEARNED MODEL

(71) Applicant: MCB Software Services Limited, Cambridge (GB)

(72) Inventors: Robert Meaker, Cambridge (GB); Liam Hayes, Cambridge (GB)

(21) Appl. No.: 16/355,167

(22) Filed: Mar. 15, 2019

(30) Foreign Application Priority Data
Mar. 16, 2018 (GB) 1804254.9

(51) Int. Cl. G06N 3/08

(52) U.S. Cl. CPC G06N 3/08

(57) ABSTRACT

An apparatus includes one or more processors configured to: receive one or more sets of data relating to a first event; input the one or more sets of data to an artificial neural network providing a learning model; receive from the learning model first output data representing a predicted duration of a task resulting from the first event; receive from the learning model second output data representing one or more predicted resources required at the end of the predicted task duration; and allocating one or more of the predicted resources available at or near the end of the predicted duration.

### FIELD

Embodiments herein relate to resource allocation using one or more learned models. Examples of resource allocation include allocating products and/or services, and may include healthcare-related products and/or services.

### BACKGROUND

In industrial and healthcare settings, there may be a need to understand when one part of a task or flow of tasks will conclude such that a subsequent task, dependent on the first part, can be performed or resources allocated for that subsequent task. The overall set of tasks may be termed a pipeline. A pipeline is a set of tasks or events, connected in series, where the output of a first task or event is the input of a second element. One or more other tasks or events may be connected to the input of the first or second elements. Some tasks or events may be performed in parallel, at least partially.

For example, in an industrial setting, it may be necessary for a first set of components to be assembled and tested in one or more first events, prior to installing the assembled first set of components onto a second set of components in one or more second events. The one or more second events are dependent on successful completion of the one or more first events. Any delay in the availability of the first set of components or their assembly will have a detrimental effect on the one or more second events, and other successive events down the line.

It would be advantageous to provide a prediction of when a first task or event will complete in order to allocate resources for delivery or performance at, or close to, the appropriate completion time.

A similar issue may arise in computer events, for example where a software resource or memory for that software resource needs to be allocated. It would be advantageous to know when to allocate the resource or memory so that it is available at the appropriate time. Reserving it beforehand may prevent other, earlier tasks, being performed.

A further similar issue may arise in healthcare, where insufficient healthcare resources are available at the time a patient is ready to leave a hospital, for example. For example, a patient able to leave hospital but needing community-based care at home may not be safely discharged until the community-based care is available. Thus, a hospital bed cannot be freed-up for another patient. This is sometimes termed a “delayed transfer of care” (DTOC) situation and can be detrimental to the patient and other patients awaiting hospitalisation.

### SUMMARY

A first aspect provides an apparatus comprising: means for receiving one or more sets of data relating to a first event; means for inputting the one or more sets of data to an artificial neural network providing a learning model; means for receiving from the learning model first output data representing a predicted duration of a task resulting from the first event; means for receiving from the learning model second output data representing one or more predicted resources required at the end of the predicted task duration; means for searching one or more databases for one or more of the predicted resources available at or near the end of the predicted duration; and means for reserving the one or more predicted resources at the one or more databases.

The apparatus may further comprise means for receiving feedback data indicative of one or both of (i) actual duration of the first event and (ii) actual resources required at the end of the predicted duration of the task, and means for updating the learning model using said feedback data.

The apparatus may further comprise: means for receiving first and second data sets relating to the first event from different external sources, and for transforming one or both of the first and second data sets into a common set of data for input to the learning model.

The means for receiving and transforming the first and second data sets may be configured to transform the data sets into one or more of a plurality of predetermined event sub-codes defining the event, which sub-codes are appropriate to the learning model.

The apparatus may further comprise means for identifying and transforming, using image recognition, one of the data sets from handwritten form to an intermediate form prior to transforming to one of the event sub-codes.

The one or more sets of data may comprise medical data may relate to a hospital admissions event for a person, wherein the first output data from the learning model represents a predicted duration of hospitalisation for the person, and wherein the second output data from the learning model represents one or more predicted care provider resources required at the end of the hospitalisation duration.

The first and second data sets may comprise computerised medical records for the person received from different respective diagnostic sources.

The means for receiving and transforming the first and second data sets may be configured to produce a plurality of predetermined diagnostic sub-codes.

The second output data from the learning model may represent a tangible care provider resource, and the reserving means is configured to order said tangible resource for delivery at or near the end of the end of the hospitalisation duration.

A second aspect provides a method, performed by one or more processors, comprising: receiving one or more sets of data relating to a first event; inputting the one or more sets of data to an artificial neural network providing a learning model; receiving from the learning model first output data representing a predicted duration of a task resulting from the first event; receiving from the learning model second output data representing one or more predicted resources required at the end of the predicted task duration; searching one or more databases for one or more of the predicted resources available at or near the end of the predicted duration; and reserving the one or more predicted resources at the one or more databases.

The method may further comprise receiving feedback data indicative of one or both of (i) actual duration of the first event and (ii) actual resources required at the end of the predicted duration of the task, and means for updating the learning model using said feedback data.

The method may further comprise receiving first and second data sets relating to the first event from different external sources, and transforming one or both of the first and second data sets into a common set of data for input to the learning model.

Receiving and transforming the first and second data sets may transform the data sets into one or more of a plurality of predetermined event sub-codes defining the event, which sub-codes are appropriate to the learning model.

The method may further comprise identifying and transforming, using image recognition, one of the data sets from handwritten form to an intermediate form prior to transforming to one of the event sub-codes.

The one or more sets of data may comprise medical data relating to a hospital admissions event for a person, wherein the first output data from the learning model represents a predicted duration of hospitalisation for the person, and wherein the second output data from the learning model represents one or more predicted care provider resources required at the end of the hospitalisation duration.

The first and second data sets may comprise computerised medical records for the person received from different respective diagnostic sources.

Receiving and transforming the first and second data sets may produce a plurality of predetermined diagnostic sub-codes.

The second output data from the learning model may represent a tangible care provider resource, and reserving may cause ordering said tangible resource for delivery at or near the end of the end of the hospitalisation duration.

Another aspect provides a computer program configured to perform the method of any of preceding method definition.

### BRIEF DESCRIPTION OF THE DRAWINGS

Example embodiments will now be described by way of non-limiting example with reference to the accompanying drawings, in which:

FIG. 1 is a schematic block diagram of an apparatus for allocating resources in accordance with one example embodiment;

FIG. 2 is a schematic block diagram of an apparatus for allocating resources in accordance with another example embodiment;

FIG. 3 is a schematic block diagram of an apparatus for allocating resources in accordance with another example embodiment;

FIG. 4 is a schematic diagram of software modules of an apparatus for allocating resources according to another example embodiment;

FIG. 5 is a schematic diagram of software operations in an apparatus for allocating resources according to another example embodiment;

FIG. 6 is a schematic diagram of components of an apparatus for allocating resources according to another example embodiment;

FIG. 7 is a flow diagram showing processing operations in a method for allocating resources according to example embodiments.

### DETAILED DESCRIPTION OF PREFERRED EMBODIMENTS

Embodiments herein relate to allocating resources based on data relating to one or more events. Embodiments involve the use of one or more artificial neural networks which provide one or more learning models to generate first output data representing a predicted duration of a task resulting from the first event, and second output data representing one or more predicted resources required at the end of the predicted task duration, i.e. at a future time. Based on these predictions, which can be generated using a single, or multiple, learning models, the one or more predicted resources can be allocated in advance of the duration end such that they are available at, or close to, the duration end. This may involve searching one or more databases associated with one or more resource providers in order to assess which providers can provide the resources at that time. If more than one resource is required, embodiments may involve generating a ‘package’ of resources and searching for a single provider that can provide all required resources at the duration end in order to minimise processing and communication effort to secure and receive the resources rather than communicating with multiple providers. In some embodiments, the searching may be done on a location basis, for example by determining the geospatial distance of the available care providers from a reference location, typically the home address of a patient, which may be a variable in selecting a suitable care package. The learning model may comprise a subroutine to search for providers within, for example, distance x before widening the scope to increasing distances.

An artificial neural network (“neural network”) is a computer system inspired by the biological neural networks in human brains. A neural network may be considered a particular kind of computational graph or architecture used in machine learning. A neural network may comprise a plurality of discrete processing elements called “artificial neurons” which may be connected to one another in various ways, in order that the strengths or weights of the connections may be adjusted with the aim of optimising the neural network's performance on a task in question. The artificial neurons may be organised into layers, typically an input layer, one or more intermediate or hidden layers, and an output layer. The output from one layer becomes the input to the next layer, and so on, until the output is produced by the final layer.

For example, in image processing, the input layer and one or more intermediate layers close to the input layer may extract semantically low-level features, such as edges and textures. Later intermediate layers may extract higher-level features. There may be one or more intermediate layers, or a final layer, that performs a certain task on the extracted high-level features, such as classification, semantic segmentation, object detection, de-noising, style transferring, super-resolution processing and so on.

Artificial neurons are sometimes referred to as “nodes”. Nodes perform processing operations, often non-linear operations. The strengths or weights between nodes are typically represented by numerical data and may be considered as weighted connections between nodes of different layers. There may be one or more other inputs called bias inputs.

There are a number of different architectures of neural network, some of which will be briefly mentioned here.

The term architecture (alternatively topology) refers to characteristics of the neural network, for example how many layers it comprises, the number of nodes in a layer, how the artificial neurons are connected within or between layers and may also refer to characteristics of weights and biases applied, such as how many weights or biases there are, whether they use integer precision, floating point precision etc. It defines at least part of the structure of the neural network. Learned characteristics such as the actual values of weights or biases may not form part of the architecture.

The architecture or topology may also refer to characteristics of a particular layer of the neural network, for example one or more of its type (e.g. input, intermediate or output layer, convolutional), the number of nodes in the layer, the processing operations to be performed by each node etc.

For example, a feedforward neural network (FFNN) is one where connections between nodes do not form a cycle, unlike recurrent neural networks. The feedforward neural network is perhaps the simplest type of neural network in that data or information moves in one direction, forwards from the input node or nodes, through hidden layer nodes (if any) to the one or more output nodes. There are no cycles or loops. Feedforward neural networks may be used in applications such as computer vision and speech recognition, and generally to classification applications.

For example, a convolutional neural network (CNN) is an architecture different from the feedforward type in that the connections between some nodes form a directed cycle, and convolution operations may take place to help correlate features of the input data across space and time, making such networks useful for applications such as handwriting and speech recognition.

For example, a recurrent neural network (RNN) is an architecture that maintains some kind of state or memory from one input to the next, making it well-suited to sequential forms of data such as text. In other words, the output for a given input depends not just on the input but also on previous inputs.

Example embodiments to be described herein may be applied to any form of neural network, providing a learning model, although examples are focussed on feedforward neural networks. The embodiments relate generally to the field of artificial intelligence (AI) which term may be considered synonymous with “neural network” or “learned model.”

When the architecture of a neural network is initialised, the neural network may operate in two phases, namely a training phase and an inference phase.

Initialised, initialisation or implementing, refers to setting up of at least part of the neural network architecture on one or more devices, and may comprise providing initialisation data to the devices prior to commencement of the training and/or inference phases. This may comprise reserving memory and/or processing resources at the particular device for the one or more layers, and may for example allocate resources for individual nodes, store data representing weights, and storing data representing other characteristics, such as where the output data from one layer is to be provided after execution. Initialisation may be incorporated as part of the training phase in some embodiments. Some aspect of the initialisation may be performed autonomously at one or more devices in some embodiments.

In the training phase, the values of the weights in the network may be determined. Initially, random weights may be selected or, alternatively, the weights may take values from a previously-trained neural network as the initial values. Training may involve supervised or unsupervised learning. Supervised learning involves providing both input and desired output data, and the neural network then processes the inputs, compares the resulting outputs against the desired outputs, and propagates the resulting errors back through the neural network causing the weights to be adjusted with a view to minimising the errors iteratively. When an appropriate set of weights are determined, the neural network is considered trained. Unsupervised, or adaptive training, involves providing input data but not output data. It is for the neural network itself to adapt the weights according to one or more algorithms. However, described embodiments are not limited by the specific training approach or algorithm used.

Once trained, the inference phase uses the trained neural network, with the weights determined during the training stage, to perform a task and generate output. For example, a task may be to predict the duration of a real world task and one or more resources that will be required at the end of that real world task.

For this purpose, one or more sets of training data may be inputted to the neural network, the training data being historical data relating to the same or similar real-world events. The actual outcomes, i.e. durations and one or more needed resources, resulting from the event, may be fed back to the neural network in order to improve its accuracy, which feedback may be iteratively performed over time to further improve accuracy. The feedback may be provided one or more times before the duration end to update the model and to modify allocations, if needed.

Embodiments herein refer to healthcare, and in particular to predicting the duration of a hospitalisation stays based one or more sets of input data received substantially at or before the time of admittance, such as diagnostic data which may be captured from a healthcare provider and/or from diagnostic equipment. Some transformation, translation or conversion of the capture data may therefore be required to ensure that what is fed into the neural network is of a consistent format.

Embodiments may also relate to predicting one or more medical or care resources needed substantially at or after the end of the duration. Embodiments may also relate to allocating said resources, such as by searching (substantially at the start of a task) for one or more providers that have said resources available substantially at the end of the task duration, or when otherwise needed. Embodiments may also relate to reserving these resources at the allocation time such that they cannot be allocated elsewhere, unless released in the meantime. Embodiments may also relate to periodically providing feedback data such that any change in a patient's condition or diagnosis may update the allocation and may be used to further train the learning model.

Embodiments are not however limited to healthcare, and find useful application in many settings, including industrial settings whereby a technical problem is similarly solved by ensuring that technical resources are available for delivery at a later time based on modelled predictions as to what resources are required, and when, based in received data. Reserving processing and/or memory resources in a computer system is one such further example.

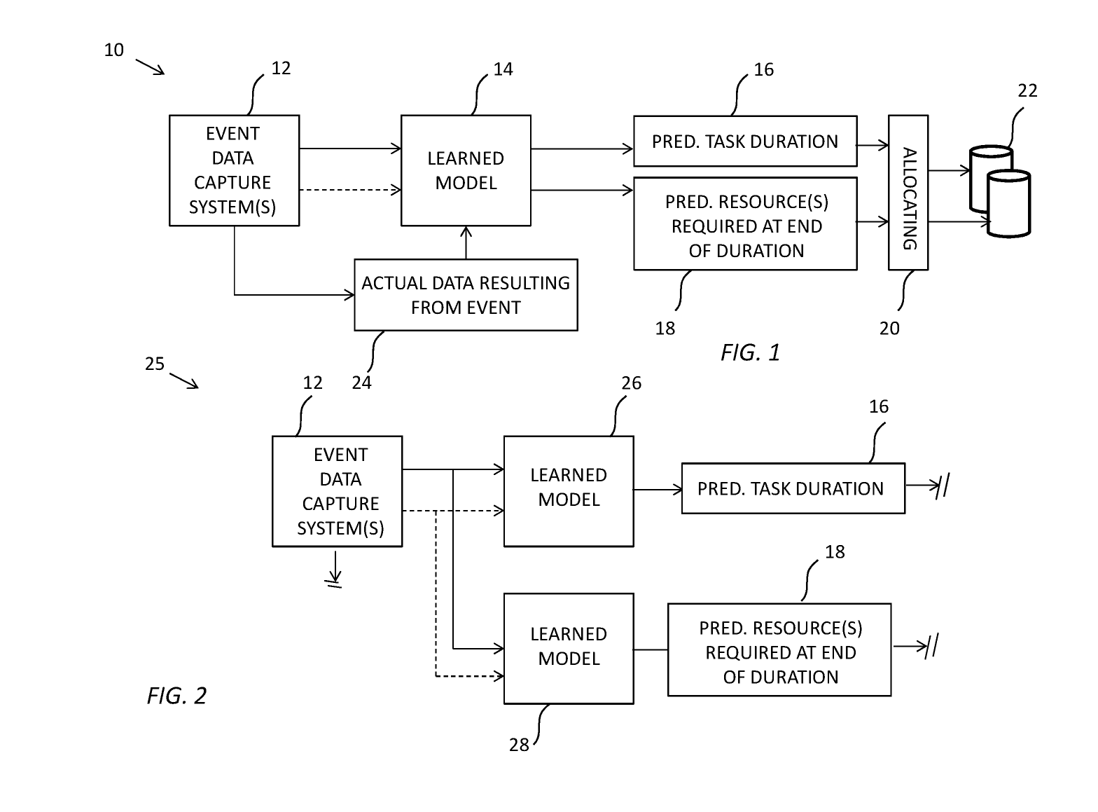

FIG. 1 is a block diagram of a system to according to an example embodiment. The system 10 comprises one or more event data capture system(s) 12 configured to capture and provide one or more input data sets to a learned model 14, which may be embodied on a neural network. The data capture system(s) 12 may comprise computer systems, tablet computers, smartphones, laptops, sensors or any other processing system(s) which can receive data relating to an event. For example, one data capture system 12 may capture data from a patient's general practitioner (GP) as one source, and another data capture system 12′ may capture data from the patient's hospital doctor at the time of admittance, as another source. The event in this example may comprise a medical event. The learned model 14, which is assumed to have been trained on a range of medical events, may produce from the received data a predicted task duration 16, for example a likely duration of hospitalisation, and one or more predicted medical or care resources 18 needed at the end of that duration, which may tangible and/or non-tangible resources.

Based on these predicted data sets, an allocating system 20 may allocate at the time of admittance or initial processing said one or more resources for delivery substantially at, or after, the predicted duration end. This may be by means of the allocating system 20 searching one or more databases 22 associated with respective care providers, identifying available resources at the appropriate time, and reserving them such that they cannot be allocated elsewhere so long as the allocation remains valid. One or more calendar or calendar-like systems may be utilised for this purpose.

A further module 24 may provide actual data resulting from the initial event. In this respect, at a later time, new data may be received from the event data capture system(s) 12, or different event data capture system(s), providing an update as to the actual progress of the task which may affect the predicted duration 16 and/or required resources 18. For example, a patient that makes quicker (or slower) than-expected progress in hospital may result in new input data which the learned model 14 uses to produce updated predictions. This may result in a reduction (or increase) in the predicted duration and/or a reduction (or increase) in the number of resources required. These updated predictions may cause the allocating system 20 to change current allocations accordingly, which may free-up resources for others. All updated data may be fed-back to the learned model 14 to improve its accuracy in accordance with known methods.

It will be appreciated that similar advantages may be offered in other technical fields.

FIG. 2 is a block diagram of a system 10 according to another example embodiment. FIG. 2 is similar to FIG. 1 save for using two different learned models 26, 28 for generating the predicted duration and predicted resources respectively. Any number of learned models 26 may be appropriate.

Generally speaking, the one or more learned models 14, 26, 28 in FIGS. 1 and 2 may be trained on a large amount of data relating to a wide range of tasks resulting from a wide range of events. For example, in the healthcare case, the one or more learned models 14, 26, 28 may be trained firstly to classify received data into one or more predetermined codes relating to symptoms and/or diagnoses. The one or more learned models 14, 26, 28 may take into account other factors, such as the patient's age, medical history, height, weight, body mass index (BMI), family history etc. in order to train and therefore predict the length of hospitalisation and resources needed afterwards. For example, a younger patient being hospitalised for a condition “A” may require less time in hospital and less resources than an older patient being hospitalised also for condition “A”. Therefore, the one or more learned models 14, 26, 28 may take multiple factors into account, and may be trained accordingly.

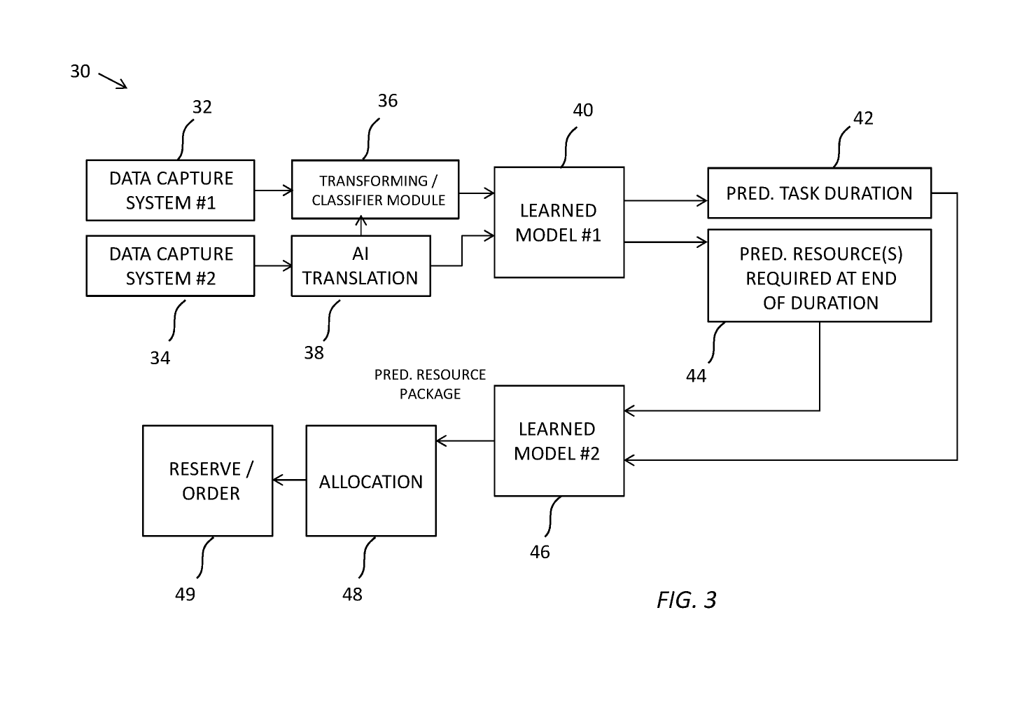

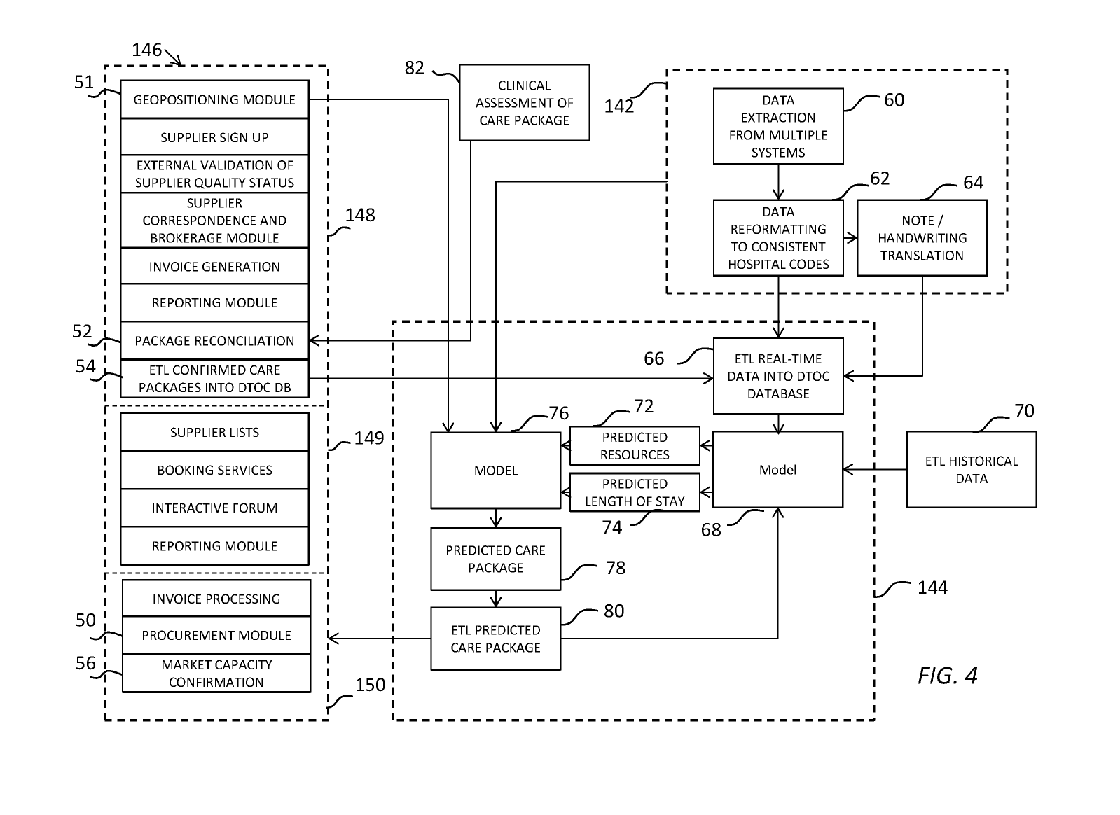

FIG. 3 is a block diagram of a system 30 according to another example embodiment. First and second data capture systems 32, 34 are provided for capturing event data from different sources. Associated with the first data capture system 32 is a transformation or classifier module 36 for converting or transforming the received data into consistent codes appropriate to a learned model 40. Thus, in the healthcare example, individual medical conditions may have respective codes and/or individual symptoms and other characteristics such as age etc. may have respective codes. The classification into consistent codes may itself use a learned model. Associated with the second data capture system 34 is an AI translation module 38 which may convert handwritten text, e.g. healthcare provider notes, into text (e.g. ASCII text) which may then be classified into the respective codes for input into the learned model 40, or may be passed to the transformation or classifier module 36 which performs said action.

The learned model 40 may perform the same function as described above with respect to FIGS. 1 and 2, and produces from the received and classified data a predicted task duration 42 and a prediction of resources required at the end of said predicted duration 44.

These two sets of prediction data 42, 44 may be provided to a further learned model 46 which, based on the combination, generates a predicted resource package based on previous trained examples of such combinations of duration and needed resources. The predicted resource package may then be provided to an allocation module 48 which searches through one or more external resource provider databases in order to broker the predicted resource package for implementation at the predicted time. This may involve reserving and/or ordering the resources.

In some embodiments, the allocation module 48 is configured first to search for a single resource provider that can offer, i.e. has availability, to provide all resources in the resource package at the required time. In this way, processing and communication effort is minimised, as are other tasks. If this is not possible, the allocation module 48 may be configured to provide all resources through only two resource providers, and so on iteratively in order to minimise the number of resource providers. A reserve/order module 49 receives the result from the allocation module 48.

FIG. 4 is a schematic diagram of software-level modules which may be used in example embodiments. The modules are arranged into three groups, namely a real-time data capture group 142, a DTOC group 144 and a web server group 146. Each group 142, 144, 146 may be implemented on a separate computer system, platform or other arrangement. The groups 142, 144, 146 may be remote from one another.

Referring first to the web server group 146, a number of different functional modules are provided, relating to the allocation stage or module mentioned previously. A first part 148 relates to background operations, and includes one or more of: a geopositioning module 51, a supplier sign-up module, an external validation of supplier quality status module, an invoice generation module, a reporting module, a package reconciliation module 52 and an extract, transform and load (ETL) confirmed care packages into DTOC database (“ETL to DTOC”) module 54 to feed into the DTOC group 144. The geo positioning module 51 may allow searching to be done on a location basis, for example by determining the geospatial distance of available care providers from a reference location, typically a home location of a patient, which may be a variable in selecting a suitable care package. The learning model may comprise a subroutine to search for providers within a given area, for example, distance x from the home location, before widening the scope to increasing distances. The ETL to DTOC module 54 may produce data which is fed back to the DTOC group 144. A second part 149 relates to commissioner operations, and includes one or more of: one or more supplier lists module, a booking services module, an interactive forum module, and a reporting module. A third part 150 relates to supplier operations, and includes one or more of an invoice processing module, a procurement module and a market capacity confirmation module 56.

Referring to the real-time data capture group 142, this comprises a data extraction module 60, a data reformatting module 62 and a note/handwriting translation module 64. The data extraction module 60 may be configured to receive or extract data from one or more sources, such as from one or more of a GP computer system, a hospital admissions system, a paramedic system etc. The data extraction module 6o may be remote from the other modules in some embodiments. The output from the data extraction module 6o is provided to the data formatting module 62 which, as described previously, is configured to convert the received or extracted data into a consistent predetermined format, possibly by checking one or more of a plurality of diagnostic or symptom classes. In some embodiments, AI may be used to classify. The consistent predetermined format is useful for input to the later learned model. The translation module 64 may receive from the data formatting module 62 any received data that cannot be classified, e.g. due to it being in handwritten form. In such a case, an image of the handwritten note or document, or sections thereof, may be processed using, e.g. handwriting recognition software (which may use a learned model), to convert the image data into, for example, ASCII text which may then be fed back to the data reformatting module 62 or may be passed directly to the DTOC group 144 as shown in the Figure.

Referring to the DTOC group 144, this comprises an ETL module 66 for passing the received, and classified, real-time data into a DTOC database. A first learning model 68 then generates the predicted resources and predicted length of stay data sets 72, 74. These data sets 72, 74 are then passed to another, second learning model 76 which generates or designs a combined care package 78 based on the combination of data sets 72, 74. Another ETL module 80 then passes the predicted care package to the procurement module 50 for allocation.

The first model 68 may also be fed historical (not real-time) data from an ETL historical data module 70, which may comprise one or more sets of training data for training said first model. The way in which the historical data is captured may use modules similar to those shown in the real-time data capture group 142, albeit the data being stored for later use rather than provided in real-time.

In some embodiments, further real-time extracted data may be received from the real-time data capture group 142, for example at a subsequent time during the patient's hospitalisation. This updated data may be fed to the model 76 to update it, with the aim of improving the model.

In some embodiments, a further module 82 may provide data representing a clinical assessment of the predicted care package as allocated. This may provide a means of human verification that the allocated care package is appropriate. Confirmation that the allocated care package is appropriate, or confirmation of one or more changes, may be fed back to the DTOC group 144, for model updating.

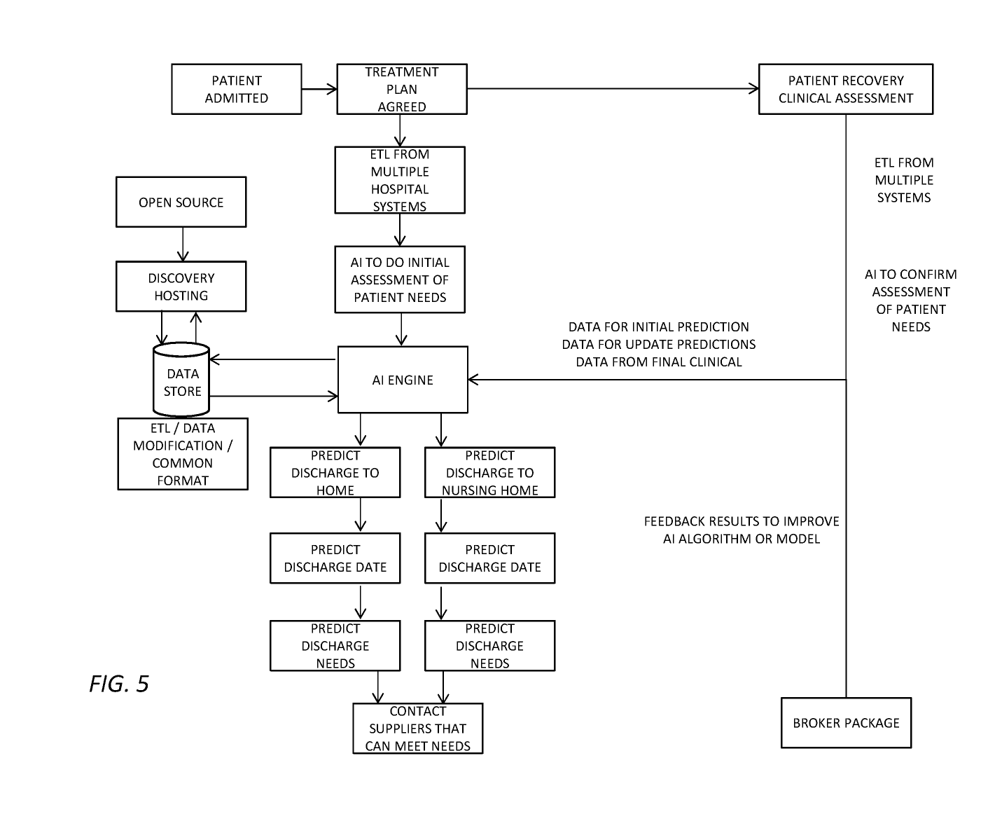

FIG. 5 is a software schematic for implementing the FIG. 4 system.

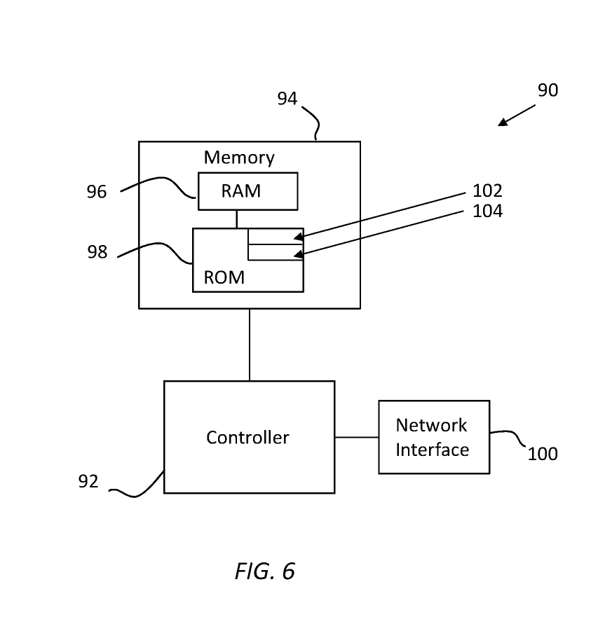

FIG. 6 is a schematic diagram of hardware components 90 for implementing any one or more of the functional components of the real-time data capture group 142, the DTOC group 144 and the web server group 146.

The components 90 may comprise a controller 92, a memory 94 closely coupled to the controller and comprised of a RAM 96 and ROM 98 and a network interface 100. It may additionally, but not necessarily, comprise a display and hardware keys. The controller 92 may be connected to each of the other components to control operation thereof. The term memory 94 may refer to a storage space.

The network interface 100 may be configured for connection to a network 21, e.g. to enable data communications between the real-time data capture group 42, the DTOC group 44 and the web server group 46. An antenna (not shown) may be provided for wireless connection, which may use WiFi, 3GPP NB-IOT, and/or Bluetooth, for example.

The memory 94 may comprise a hard disk drive (HDD) or a solid state drive (SSD). The ROM 98 of the memory 94 stores, amongst other things, an operating system 102 and may store one or more software applications 104. The RAM 96 is used by the controller 92 for the temporary storage of data. The operating system 102 may contain code which, when executed by the controller 92 in conjunction with the RAM 96, controls operation of each of the hardware components.

The controller 92 may take any suitable form. For instance, it may be a microcontroller, plural microcontrollers, a processor, plural processors, or processor circuitry.

In some example embodiments, the components 90 may also be associated with external software applications. These may be applications stored on a remote server device and may run partly or exclusively on the remote server device. These applications may be termed cloud-hosted applications or data. The components 90 may be in communication with the remote server device in order to utilize the software application stored there.

The processing operations to be described below may be performed by the one or more software applications 104 provided on the memory 94, or on hardware, firmware or a combination thereof.

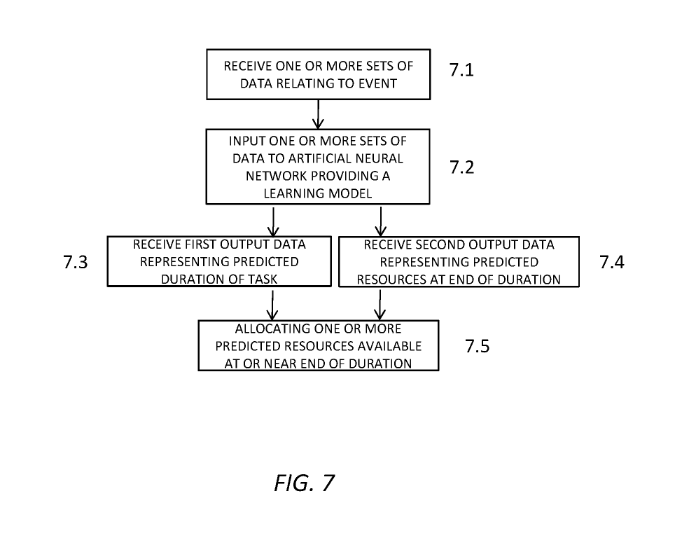

FIG. 7 is a flow diagram showing example operations that may be performed by the components shown in FIG. 6. The operations may be performed in hardware, software or a combination thereof. One or more operations may be omitted. The number of operations is not necessarily indicative of the order of processing.

A first operation 7.1 may comprise receiving one or more sets of data relating to an event. A second operation 7.2 may comprise inputting one or more sets of the received data to an artificial neural network providing a learning model. A third operation 7.3 may comprise receiving first output data representing a predicted duration of a task. A fourth operation 7.4 may comprise receiving second output data representing a predicted set or resources at the end of the duration. A fifth operation 7.5 may comprise allocating one or more predicted resources available at or near the end of the duration.

It will be appreciated that certain operations may be omitted or re-ordered. The numbering of the operations is not necessarily indicative of their processing order.

A tangible resource may comprise medical or care equipment, such as a wheelchair, handrail, medication, a room in a nursing or care home etc. A non-tangible resource may comprise a service such as home nursing, X-rays, MRI scanning etc. Such resources may be provided by external providers not necessarily being part of the same hospital or health service.

Embodiments herein, if employed in healthcare, enable reduction of delayed transfers of care (DTOC) from hospitals, currently an issue of escalating concern. The resultant effect of these delays on patients is poorer outcomes, and for older patients in particular, increases the risk of readmission. Some embodiments may enable predicting the outcome (and related care needs of patients) at the point of admission to identify suitable care homes and/or domiciliary care with capacity, updating as the patients' circumstances change. Some embodiments may automate a number of stages to speed up the discharge planning process; effective discharge planning is essential to ensure that people have the care and support they need in place before they are discharged, else they risk deteriorating and being readmitted to hospital. This may be achieved using machine learning algorithms for translating acute information into actionable data in real time. Allocating stages aim to ensure capacity is available to meet a patients' needs when medically fit for discharge. Care packages may be established in draft form immediately upon an acute admission. Where market capacity is limited, brokers have time to source alternative providers, during the treatment phase. Using machine learning and building on existing products in the manner described helps provide an end-to-end solution for discharge planning. Currently, the assessment process does not start until treatment is completed, and therefore the market (care homes and domiciliary agencies) is not aware of the needs and cannot plan accordingly. Embodiments employ machine learning in determining appropriate care packages at the point of admission and identifying capacity to meet the needs at the estimated time of discharge.

Other embodiments, founded on similar principles, may be applied to industrial and/or computational applications to allocate resources, e.g. industrial components, software resources, computer memory resources, based on machine learning. The embodiments take event data as input and predicts therefrom what resources (e.g. components or software or memory resources) may be needed at a given future time, and allocates these resources at that time for use at the required future time.

Implementations of any of the above described blocks, apparatuses, systems, techniques or methods include, as non-limiting examples, implementations as hardware, software, firmware, special purpose circuits or logic, general purpose hardware or controller or other computing devices, or some combination thereof. Some example embodiments may be implemented in the cloud and utilize virtualized modules.

Example embodiments of the present invention may be implemented in software, hardware, application logic or a combination of software, hardware and application logic. The software, application logic and/or hardware may reside on memory, or any computer media. In an example embodiment, the application logic, software or an instruction set is maintained on any one of various conventional computer-readable media. In the context of this document, a “memory” or “computer-readable medium” may be any non-transitory media or means that can contain, store, communicate, propagate or transport the instructions for use by or in connection with an instruction execution system, apparatus, or device, such as a computer.

Reference to, where relevant, “computer-readable storage medium”, “computer program product”, “tangibly embodied computer program” etc., or a “processor” or “processing circuitry” etc. should be understood to encompass not only computers having differing architectures such as single/multi-processor architectures and sequencers/parallel architectures, but also specialized circuits such as field programmable gate arrays FPGA, application specify circuits ASIC, signal processing devices and other devices. References to computer program, instructions, code etc. should be understood to express software for a programmable processor firmware such as the programmable content of a hardware device as instructions for a processor or configured or configuration settings for a fixed function device, gate array, programmable logic device, etc.

As used in this application, the term “means” refers to all of the following: (a) hardware-only circuit implementations (such as implementations in only analogue and/or digital circuitry) and (b) to combinations of circuits and software (and/or firmware), such as (as applicable): (i) to a combination of processor(s) or (ii) to portions of processor(s)/software (including digital signal processor(s)), software, and memory(ies) that work together to cause an apparatus, such as a server, to perform various functions) and (c) to circuits, such as a microprocessor(s) or a portion of a microprocessor(s), that require software or firmware for operation, even if the software or firmware is not physically present.

In this brief description, reference has been made to various examples. The description of features or functions in relation to an example indicates that those features or functions are present in that example. The use of the term ‘example’ or ‘for example’ or ‘may’ in the text denotes, whether explicitly stated or not, that such features or functions are present in at least the described example, whether described as an example or not, and that they can be, but are not necessarily, present in some of or all other examples. Thus ‘example’, ‘for example’ or ‘may’ refers to a particular instance in a class of examples. A property of the instance can be a property of only that instance or a property of the class or a property of a sub-class of the class that includes some but not all of the instances in the class. It is therefore implicitly disclosed that a features described with reference to one example but not with reference to another example, can where possible be used in that other example but does not necessarily have to be used in that other example.

Although embodiments of the present invention have been described in the preceding paragraphs with reference to various examples, it should be appreciated that modifications to the examples given can be made without departing from the scope of the invention as claimed.

Features described in the preceding description may be used in combinations other than the combinations explicitly described.

Although functions have been described with reference to certain features, those functions may be performable by other features whether described or not.

Although features have been described with reference to certain embodiments, those features may also be present in other embodiments whether described or not.

Whilst endeavoring in the foregoing specification to draw attention to those features of the invention believed to be of particular importance it should be understood that the Applicant claims protection in respect of any patentable feature or combination of features hereinbefore referred to and/or shown in the drawings whether or not particular emphasis has been placed thereon.

1. Apparatus comprising:
one or more processors; and
one or more memories storing instructions, that, when executed by the one or more processors, cause the apparatus to perform a computer-implemented method of:
receiving one or more sets of data relating to a first event;
inputting the one or more sets of data to an artificial neural network providing a learning model;
receiving from the learning model first output data representing a predicted duration of a task resulting from the first event;
receiving from the learning model second output data representing one or more predicted resources required at the end of the predicted task duration; and
allocating one or more of the predicted resources available at or near the end of the predicted duration.

2. The apparatus of claim 1, wherein the allocating comprises searching one or more databases for one or more of the predicted resources available at or near the end of the predicted duration; and reserving the one or more predicted resources at the one or more databases.

3. The apparatus of claim 1, further comprising receiving feedback data indicative of one or both of (i) actual duration of the first event and (ii) actual resources required at the end of the predicted duration of the task, and means for updating the learning model using said feedback data.

4. The apparatus of claim 1, further comprising:
receiving first and second data sets relating to the first event from different external sources, and transforming one or both of the first and second data sets into a common set of data for input to the learning model.

5. The apparatus of claim 4, wherein receiving and transforming the first and second data sets transforms the data sets into one or more of a plurality of predetermined event sub-codes defining the event, which sub-codes are appropriate to the learning model.

6. The apparatus of claim 4, further comprising identifying and transforming, using image recognition, one of the data sets from handwritten form to an intermediate form prior to transforming to one of the event sub-codes.

7. The apparatus of claim 1, wherein the one or more sets of data comprise medical data relating to a hospital admissions event for a person, wherein the first output data from the learning model represents a predicted duration of hospitalisation for the person, and wherein the second output data from the learning model represents one or more predicted care provider resources required at the end of the hospitalisation duration.

8. The apparatus of claim 7, wherein the first and second data sets comprise computerised medical records for the person received from different respective diagnostic sources.

9. The apparatus of claim 8, further comprising receiving first and second data sets relating to the first event from different external sources, and transforming one or both of the first and second data sets into a common set of data for input to the learning model, wherein receiving and transforming the first and second data sets produces a plurality of predetermined diagnostic sub-codes.

10. The apparatus of claim 7, wherein the second output data from the learning model represents a tangible care provider resource, and the reserving means is configured to order said tangible resource for delivery at or near the end of the end of the hospitalisation duration.

11. A computer-implemented method, performed by one or more processors, comprising:
receiving one or more sets of data relating to a first event;
inputting the one or more sets of data to an artificial neural network providing a learning model;
receiving from the learning model first output data representing a predicted duration of a task resulting from the first event;
receiving from the learning model second output data representing one or more predicted resources required at the end of the predicted task duration; and
allocating one or more of the predicted resources available at or near the end of the predicted duration.

12. The computer-implemented method of claim 11, wherein allocating comprises searching one or more databases for one or more of the predicted resources available at or near the end of the predicted duration; and reserving the one or more predicted resources at the one or more databases.

13. The computer-implemented method of claim 12, further comprising receiving feedback data indicative of one or both of (i) actual duration of the first event and (ii) actual resources required at the end of the predicted duration of the task, and means for updating the learning model using said feedback data.

14. The computer-implemented method of claim 12, further comprising:
receiving first and second data sets relating to the first event from different external sources, and transforming one or both of the first and second data sets into a common set of data for input to the learning model.

15. The computer-implemented method of claim 14, wherein receiving and transforming the first and second data sets transforms the data sets into one or more of a plurality of predetermined event sub-codes defining the event, which sub-codes are appropriate to the learning model.

16. The computer-implemented method of claim 14, further comprising identifying and transforming, using image recognition, one of the data sets from handwritten form to an intermediate form prior to transforming to one of the event sub-codes.

17. The computer-implemented method of claim 12, wherein the one or more sets of data comprise medical data relating to a hospital admissions event for a person, wherein the first output data from the learning model represents a predicted duration of hospitalisation for the person, and wherein the second output data from the learning model represents one or more predicted care provider resources required at the end of the hospitalisation duration.

18. The computer-implemented method of claim 17, further comprising:
receiving first and second data sets relating to the first event from different external sources, and transforming one or both of the first and second data sets into a common set of data for input to the learning model.
wherein the first and second data sets comprise computerised medical records for the person received from different respective diagnostic sources.

19. One or more non-transitory computer-readable mediums comprising instructions stored thereon, which when executed by one or more processors configured the one or more processors to perform a computer-implemented method comprising:
receiving one or more sets of data relating to a first event;
inputting the one or more sets of data to an artificial neural network providing a learning model;
receiving from the learning model first output data representing a predicted duration of a task resulting from the first event;
receiving from the learning model second output data representing one or more predicted resources required at the end of the predicted task duration; and
allocating one or more of the predicted resources available at or near the end of the predicted duration.
